(19) United States
(12) Patent Application Publication
(10) Pub. No.: US 2004/0254248 A1
(43) Pub. Date: Dec. 16, 2004

(54) Method of treatment and/or prophylaxis of ulcers

(76) Inventor: Neil Roland McGregor, Eleebana (AU)

Correspondence Address: SUGHRUE MION, PLLC, 2100 PENNSYLVANIA AVENUE, N.W., SUITE 800, WASHINGTON, DC, 20037, US

(21) Appl. No.: 10/483,392

(22) PCT Filed: Jul. 5, 2002
(86) PCT No.: PCT/AU02/00891

(30) Foreign Application Priority Data
Jul. 10, 2001 (AU) PR 6260

(51) Int. Cl. A61K 31/19

(52) U.S. Cl. 514/574

(57) ABSTRACT

The present invention relates generally to a method for the treatment and/or prophylaxis of a condition involving one or more ulcers that affect the inside of the mouth and other mucous membranes. More particularly, the present invention is directed to a method for treating and/or reducing the likelihood of developing ulcers and particularly of the recurrence of recurrent ulcers. The present invention is particularly directed to a composition which promotes or enhances the healing of aphthouse ulcers thereby reducing at least one of the symptoms of aphthous ulcers and/or the risk of developing one or more of said symptoms.

### FIELD OF THE INVENTION

The present invention relates generally to a method for the treatment and/or prophylaxis of a condition involving one or more ulcers that affect the inside of the mouth and other mucous membranes. More particularly, the present invention is directed to a method for treating and/or reducing the likelihood of developing ulcers and particularly of the recurrence of recurrent ulcers. The present invention is particularly directed to a composition which promotes or enhances the healing of aphthous ulcers thereby reducing at least one of the symptoms of aphthous ulcers and/or the risk of developing one or more of said symptoms.

### BACKGROUND OF THE INVENTION

Bibliographic details of the publications referred to by author in this specification are collected at the end of the description.

Reference to any prior art in this specification is not, and should not be taken as an acknowledgment or any form of suggestion that this prior art is common general knowledge or forms a part of the common general knowledge in Australia or any other country.

Recurrent aphthous ulcers or canker sores are small ulcers that affect the mucous membranes inside the mouth, nasopharynx, oesophagus, gastrointestinal tract and genitalia. Orally they are usually found on the buccal and labial mucosa, gingiva and less frequently on the tongue. They may occur singly or in groups. They are twice as likely to occur in females than males and there appears to be a familial tendency. They predominantly occur in subjects of between 10 and 40 years of age.

An initial prodromal phase occurs before the ulceration occurs which is usually associated with a tingling or burning sensation followed by the development of a bulla. Following the bursting of the bulla (6-24 hours after the initiation of the lesion) an ulcer appears as a small, round depression up to 0.5 cm in diameter, surrounded by a reddened area of inflammation. The centre is often greyish-white due to a layer of fibrin and dead cells. Below this fibrin surface the tissue is raw and erythematous.

The aphthous ulcer is most painful during the first 3-4 days and the pain gradually diminishes as the lesion heals over the following 10-14 days. The lesions usually heal without scaring however if the lesions occur on the gingival margin or on the thin mucosa on the lingual of the mandible, scaring or bone exposure can occur. Recurrent aphthous ulcers frequently recur with some patients suffering many bouts of ulcers during a year.

Although the cause of recurrent aphthous ulcers is unknown, certain triggers have been identified. These include certain drugs, trauma, certain foods such as for example chocolate, walnuts, citrus fruit, tomatoes, strawberries, vinegar; and certain vitamins such as large doses of Vitamin C. Dietary deficiencies, such as for example iron, folic acid and vitamin B12 deficiencies; menstrual periods and hormonal changes have also been reported as triggers.

In some cases, the condition is self-limited and healing is spontaneous. In other cases, patients have repeated bouts of aphthous ulcers which require intervention. Several treatments have been tried including tetracycline mouth rinses and under orohesive bandages, topical corticosteroids, the cautary agent Debacterol (Rhodus & Bereuter, 1998) and the drug Aphthasol (amlexanox) (Khandwala et al., 1997a, 1997b). However, there is a need for effective treatment to promote healing, reduce the frequency of bouts and remove the pain associated with the condition.

In the work leading up to the present invention the instant inventor has developed a method for the treatment and/or prophylaxis of aphthous ulcers comprising the administration of a composition which promotes or enhances the healing of ulcers thereby reducing one or more of the symptoms of aphthous ulcers and/or the risk of developing same.

### SUMMARY OF THE INVENTION

Throughout this specification, unless the context requires otherwise the word “comprise”, and variations such as “comprises” and “comprising”, will be understood to imply the inclusion of a stated element or integer or step or group of elements or integers or steps but not the exclusion of any other element or integer or step or group of elements or integers or steps.

In a first aspect, the present invention provides a method for the treatment and/or prophylaxis of a condition involving an ulcer in a subject, said method comprising administration to said subject of a composition comprising a citrate salt and/or a succinate salt to promote or otherwise enhance the healing of said ulcer thereby reducing at least one of the symptoms of said ulcer and/or the risk of developing one or more of said symptoms.

Another aspect of the present invention provides a method for the treatment and/or prophylaxis of a condition involving an ulcer in a subject, said method comprising administration to said subject of a composition comprising a citrate salt and/or a succinate salt to reduce or inhibit viral replication associated with said ulcer thereby reducing at least one of the symptoms of said ulcer and/or the risk of developing one or more of said symptoms.

Still another aspect of the present invention provides a method for the treatment and/or prophylaxis of a condition involving an ulcer in a subject, said method comprising administration, to said subject, of a composition comprising a citrate salt and a succinate salt to promote or otherwise enhance the healing of said ulcer thereby reducing at least one of the symptoms of said ulcer and/or the risk of developing one or more of said symptoms.

Even yet another aspect of the present invention provides a method for the treatment and/or prophylaxis of a condition involving an ulcer in a subject, said method comprising administration, to said subject, of a composition comprising a citrate salt and/or a succinate salt and an amino acid selected from the group comprising valine, aspartic acid, leucine, isoleucine, alanine, lysine, taurine and asparagine, said composition being effective to promote or otherwise enhance the healing of said ulcer thereby reducing at least one of the symptoms of said ulcer and/or the risk of developing one or more of said symptoms.

Even still another aspect of the present invention provides a method for the treatment and/or prophylaxis of a condition involving an ulcer in a subject, said method comprising administration, to said subject of an effective amount of a citrate salt and/or a succinate salt, and optionally an amino acid selected from the group comprising valine, aspartic acid, leucine, isoleucine, alanine, lysine, taurine and asparagine, to promote or otherwise enhance the healing of said ulcer thereby reducing at least one of the symptoms of said ulcer and/or the risk of developing one or more of said symptoms.

Yet another related aspect of the present invention provides a composition suitable for use in the treatment and/or prophylaxis of a condition involving an ulcer in a subject, said composition comprising a citrate salt and/or a succinate salt, and optionally an amino acid selected from the group comprising valine, aspartic acid, leucine, isoleucine, alanine, lysine, taurine and asparagine, to promote or otherwise enhance the healing of said ulcer thereby reducing at least one of the symptoms of said ulcer and/or the risk of developing one or more of said symptoms.

Still another aspect of the present invention provides the use of a citrate salt and/or a succinate salt, and optionally an amino acid selected from the group comprising valine, aspartic acid, leucine, isoleucine, alanine, lysine, taurine and asparagine, in the manufacture of a medicament for the treatment and/or prophylaxis of a condition involving an ulcer in a subject.

### DETAILED DESCRIPTION OF THE INVENTION

The present invention is predicated in part on the development of a composition which effectively and rapidly promotes healing of ulcers in patients.

In a first aspect, the present invention provides a method for the treatment and/or prophylaxis of a condition involving an ulcer in a subject, said method comprising administration to said subject of a composition comprising a citrate salt and/or a succinate salt to promote or otherwise enhance the healing of said ulcer thereby reducing at least one of the symptoms of said ulcer and/or the risk of developing one or more of said symptoms.

Reference herein to the term “ulcer” or “ulceration” should be read as including reference to all conditions involving an ulcer or sore. Particularly contemplated are conditions which involve ulcers caused by or associated with viral replication and ulcers which affect a mucous membrane such for example, mucous membranes of the mouth, nasopharynx, oesophagus, gastrointestinal tract and genitalia.

Reference herein to the term “prophylaxis of a condition involving an ulcer in a subject” should be read as including reference to prevention or reduction of the risk of occurrence of a condition involving an ulcer and to prevention or reduction of the likelihood of the recurrence of a condition involving an ulcer.

In one particular embodiment, said condition is recurrent aphthous ulceration (RAU).

While not wishing to limit the present invention in any way to one particular theory or mode of action, it is proposed herein that the present composition is effective in inhibiting or at least reducing viral replication associated with the development and/or progression of conditions associated with recurrent ulceration. Viral infection and replication in the tissue near the site of the ulcer may stimulate an inflammatory response which manifest as one or more symptoms of an ulcer such as inflammation including recruitment and proliferation of T-cells and/or cell death.

Accordingly, a second aspect of the present invention provides a method for the treatment and/or prophylaxis of a condition involving an ulcer in a subject, said method comprising administration to said subject of a composition comprising a citrate salt and/or a succinate salt to reduce or inhibit viral replication associated with said ulcer thereby reducing at least one of the symptoms of said ulcer and/or the risk of developing one or more of said symptoms.

Reference herein to the term “symptoms of said ulcer” should be read as a reference to any of the symptoms of an ulcer including, a burning or tingling sensation or pain at or near said ulcer, a local and/or general inflammatory response, or the formation and progression of an ulcer. Those skilled in the art will be familiar with the symptoms of ulcers and conditions associated with the formation of ulcers.

Reference herein to the term “promote or otherwise enhance the healing” includes reference to improving the rate and quality of healing of the ulcer such as the rate of epithelialisation but also relates, as understood by those skilled in this art, to reducing the likelihood of recurrence of an ulcer.

Reference herein to the term “citrate salt” includes reference to any citrate salt which is effective in reducing at least one of the symptoms of ulceration and/or the risk of developing one or more of said symptoms.

Preferably, said citrate salt is selected from the group comprising sodium citrate, potassium citrate or magnesium citrate and the like. The choice of the cation will depend on factors such as solubility of the citrate salt and whether or not it is desirable to include a particular cation. For example, high levels of potassium may affect kidney function.

Reference herein to the term “succinate salt” includes reference to any succinate salt which is effective in reducing or enhancing the reduction of at least one of the symptoms of ulceration and/or the risk of developing one or more of said symptoms.

Preferably, said succinate salt is selected from the group comprising sodium succinate, potassium succinate, calcium succinate or magnesium succinate and the like. The choice of the cation will depend on factors such as solubility of the succinate salt and whether or not it is desirable to include a particular cation. For example, high levels of potassium may affect kidney function.

In another aspect, the present invention provides a method for the treatment and/or prophylaxis of a condition involving an ulcer in a subject, said method comprising administration to said subject of a composition comprising a citrate salt and a succinate salt to promote or otherwise enhance the healing of said ulcer thereby reducing at least one of the symptoms of said ulcer and/or the risk of developing one or more of said symptoms.

In the exemplified embodiment, the present inventors have further enhanced the healing promoting properties of the composition by including therein one or more amino acids selected from the group comprising, valine, aspartic acid, leucine, isoleucine, alanine, lysine, taurine and asparagine.

According to a further embodiment therefore, the present invention provides a method for the treatment and/or prophylaxis of a condition involving an ulcer in a subject, said method comprising administration, to said subject, of a composition comprising a citrate salt and/or succinate salt, and an amino acid selected from the group comprising valine, aspartic acid, leucine, isoleucine, alanine, lysine, taurine and asparagine, said composition being effective to promote or otherwise enhance the healing of said ulcer thereby reducing at least one of the symptoms of said ulcer and/or the risk of developing one or more of said symptoms.

Reference herein to “amino acid” includes reference to derivatives, homologues, analogues and mimetics thereof which will be well known to those skilled in the art. Taurine, for example, is an analogue of b-alanine. Chemical analogues of the subject amino acids contemplated herein include, but are not limited to, modification to side chains such as amino or carboxyl groups.

In yet another aspect the present invention provides a method for the treatment and/or prophylaxis of a condition involving an ulcer in a subject, said method comprising administration, to said subject of an effective amount of a citrate salt and/or a succinate salt, and optionally an amino acid selected from the group comprising valine, aspartic acid, leucine, isoleucine, alanine, lysine, taurine and asparagine, to promote or otherwise enhance the healing of said ulcer thereby reducing at least one of the symptoms of said ulcer and/or the risk of developing one or more of said symptoms.

Another embodiment of the present invention provides a composition suitable for use in the treatment and/or prophylaxis of a condition involving an ulcer in a subject, said composition comprising a citrate salt and/or a succinate salt to promote or otherwise enhance the healing of said ulcer thereby reducing at least one of the symptoms of said ulcer and/or the risk of developing one or more of said symptoms.

Still another embodiment of the present invention provides a composition when used in the treatment and/or prophylaxis of a condition involving an ulcer in a subject, said composition comprising a citrate salt and/or a succinate salt to promote or otherwise enhance the healing of said ulcer thereby reducing at least one of the symptoms of said ulcer and/or the risk of developing one or more of said symptoms.

Components of the composition may be from any convenient source. For example, they may be in purified form or they maybe in the form of herbs or preferably an extract of herbs or horticultural or botanical equivalents of herbs or chemical or functional equivalents of the herb extract.

Oral administration of the composition of the present invention is contemplated although delivery may be by any convenient means such as intravenous, intranasal, intraperitoneal, subcutaneous, intradermal, topical, suppository routes or implanting (slow-release molecules).

Pharmaceutical forms of the composition may be suitable for injectable use such as sterile aqueous solutions and sterile powders for the extemporaneous preparation of sterile injectable solutions or dispersions.

The composition must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof and vegetable oils. The proper fluidity can be maintained, for example, by the use of a coating such as lecithin. The prevention of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

The compositions may be orally administered, for example, with an inert diluent or with an assimilable edible carrier, or it may be enclosed in hard or soft shell gelatin capsule, or it may be compressed into tablets, or it may be in powdered form or incorporated directly with the food of the diet. For oral therapeutic and/or prophylactic administration, the active compound may be incorporated with excipients and used in the form of ingestible tablets, buccal tablets, troches, capsules, elixirs, suspensions, syrups, wafers, and the like.

A broad range of doses may be applicable depending on the subject, severity of condition and proposed route and medium for administration. The amount of active compound in such therapeutically useful compositions is such that a suitable dosage will be obtained. Preferred compositions according to the present invention are prepared so that an oral dosage unit form contains between about 0.01 μg and about 2000 mg of active compound. Alternative amounts include between about 1.0 μg and about 1500 ng, between about 1 μg and about 1000 mg and between about 10 μg and about 500 mg.

The tablets, troches, pills, capsules and the like may also contain the components as listed hereafter: A binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active ingredient or ingredients are compounded for convenient and effective administration in effective amounts with a suitable pharmaceutically acceptable carrier in dosage unit form. A unit dosage form can, for example, contain the principal active compounds in amounts ranging from 0.01 μg to about 70 g/100 grams. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients. Alternatively, amounts administered may be represented in terms of amounts/kg body weight. In this case, amounts range from about 0.001 μg to about 1000 mg/kg body weight may be administered. Preferred ranges include from about 50 μg to 500 mg 1 kg body weight 500 mg/kg body weight or about 0.01 μg to about or above 0.1 μg to about 250 mg/kg body weight are contemplated by the present invention.

Prophylactic administration is clearly contemplated herein. Preferably, the composition is administered at an early phase of ulcer development, for example at the tingling or burning phase. Alternatively, the composition is administered when it is believed that one of the triggers for development of ulcers has been taken or experienced. For example triggers may be ingestion of food such as walnuts, chocolate, vinegar, citrus fruit, tomatoes and strawberries; large doses of Vitamin C; certain drugs, trauma, puberty and other times of hormonal fluctuation. The dose and frequency of dosing is determined by a number of factors including body weight, severity and location of ulcers and frequency of recurrence of ulcers.

The present invention is now further described with reference to the following non-limiting examples.

### EXAMPLE 1

The following composition was tested in subjects:

| | |
|---|---|
| Compound | mg/gram |

| | | |
|---|---|---|
| Sodium Citrate | 488.8 | mg |
| L-Isoleucine | 39.0 | mg |
| L-Valine | 39.0 | mg |
| L-Asparagine | 39.0 | mg |
| L-Alanine | 26.0 | mg |
| L-Leucine | 26.0 | mg |
| L-Glycine | 26.0 | mg |
| L-Serine | 26.0 | mg |
| L-Threonine | 26.0 | mg |
| L-Tyrosine | 19.5 | mg |
| L-Phenylalanine | 19.5 | mg |
| L-Glutamine | 19.5 | mg |
| L-Proline | 19.5 | mg |
| L-Tryptophan | 19.5 | mg |
| L-Lysine | 19.5 | mg |
| Taurine | 19.5 | mg |
| L-Methionine | 13.0 | mg |
| L-Arginine | 13.0 | mg |
| L-Histidine | 13.0 | mg |
| L-Cystine | 13.0 | mg |
| Magnesium aspartate | 39.0 | mg |
| Calcium succinate | 13.0 | mg |
| Nicotinamide | 7.8 | mg |
| d-alpha Tocopheryl acetate | 5.2 | mg |
| Ferrous fumarate | 5.2 | mg |
| Alpha-Lipoic acid | 2.6 | mg |
| Calcium Pantothenate | 1.3 | mg |
| Riboflavine | 0.8 | mg |
| Thiamine | 0.5 | mg |
| Betacarotene | 0.2 | mg |
| Biotin | 10 | mcg |
| Cholecalciferol | 10 | mcg |
| Cyanocobalamin | 10 | mcg |

### EXAMPLE 2

A female subject aged 12 years, who has multiple aphthous ulcers for a period of 4 weeks, took a 300 mg capsule of the supplement of Example 1 and by the following morning the aphthous ulcers appeared epithelialised and the patient was pain free. By the next day the ulcers had epithelialised.

### EXAMPLE 3

A male subject aged 35 years, who has multiple bouts of aphthous ulcers throughout their life and had a current bout of ulcers for a period of 2 weeks, took a 300 mg capsule of the supplement of Example 1 and by the following morning the aphthous ulcers had epithelialised and the patient was pain free. By the next day the ulcers had epithelialised.

### EXAMPLE 4

The following composition is tested in subjects,

| | |
|---|---|
| Compound | mg/gram |

| | | |
|---|---|---|
| Sodium Citrate | 488.8 | mg |
| L-Isoleucine | 39.0 | mg |
| L-Valine | 39.0 | mg |
| L-Asparagine | 39.0 | mg |
| L-Alanine | 26.0 | mg |
| L-Leucine | 26.0 | mg |
| L-Glycine | 26.0 | mg |
| L-Serine | 26.0 | mg |
| L-Threonine | 26.0 | mg |
| L-Tyrosine | 19.5 | mg |
| L-Phenylalanine | 19.5 | mg |
| L-Glutamine | 19.5 | mg |
| L-Proline | 19.5 | mg |
| L-Tryptophan | 19.5 | mg |
| L-Lysine | 19.5 | mg |
| Taurine | 19.5 | mg |
| L-Histidine | 13.0 | mg |
| L-Cystine | 13.0 | mg |
| Magnesium aspartate | 39.0 | mg |
| Calcium succinate | 13.0 | mg |
| Nicotinamide | 7.8 | mg |
| d-alpha Tocopheryl acetate | 5.2 | mg |
| Ferrous fumarate | 5.2 | mg |
| Alpha-Lipoic acid | 2.6 | mg |
| Calcium Pantothenate | 1.3 | mg |
| Riboflavine | 0.8 | mg |
| Thiamine | 0.5 | mg |
| Betacarotene | 0.2 | mg |
| Biotin | 10 | mcg |
| Cholecalciferol | 10 | mcg |
| Cyanocobalamin | 10 | mcg |

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Rhodus N L, Bereuter J. An evaluation of a chemical cautery agent and an anti-inflammatory ointment for the treatment of recurrent aphthous stomatitis: a pilot study. Quint Internat; 29:769-773,1998.

Khandwala A, Van Inwegen R G, Charney M R, Alfano M C. 5% amlexanox oral paste, a new treatment for recurrent minor aphthous ulcers: II. Pharmacokinetics and demonstration of clinical safety. Oral Surg Oral Med Oral Path Oral Radiol Endo; 83:231-238, 1997a.

Khandwala A. Van Inwegen R G. Alfano M C. 5% amlexanox oral paste, a new treatment for recurrent minor aphthous ulcers: I. Clinical demonstration of acceleration of healing and resolution of pain. Oral Surg Oral Med Oral Path Oral Radiol Endo; 83:222-230, 1997b.

What is claimed is:

1. A method for the treatment and/or prophylaxis of a condition involving an ulcer in a subject, said method comprising administration to said subject of a composition comprising a citrate salt and/or a succinate salt to promote or otherwise enhance the healing of said ulcer thereby reducing at least one of the symptoms of said ulcer and/or the risk of developing one or more of said symptoms.

2. A method according to claim 1 wherein said composition comprises a citrate salt and a succinate salt.

3. A method according to claim 1 wherein said composition further comprises an amino acid selected from valine, aspartic acid, leucine, isoleucine, alanine, lysine, taurine or asparagine.

4. A method according to claim 1 wherein said condition involving an ulcer is recurrent aphthous ulceration.

5. A method according to claim 1 wherein said subject is a human subject.

6. A method according to claim 1 wherein said citrate salt is sodium citrate, potassium citrate or magnesium citrate.

7 A method according to claim 6 wherein said citrate is sodium citrate.

8. A method according to claim 1 wherein said succinate salt is sodium succinate, potassium succinate, calcium succinate or magnesium succinate.

9. A method according to claim 8 wherein said succinate is calcium succinate.

10. Use of a citrate salt and/or a succinate salt, and an amino acid selected from valine, aspartic acid, leucine, isoleucine, alanine, lysine, taurine and asparagine, in the manufacture of a medicament for the treatment and/or prophylaxis of a condition involving an ulcer in a subject.

11. A composition for use in the treatment and/or prophylaxis of a condition involving an ulcer in a subject, said composition comprising a citrate salt and/or a succinate salt optionally together with one or more amino acids selected from valine, aspartic acid, leucine, isoleucine, alanine, lysine, taurine and asparagine.
